(19) United States
(12) Patent Application Publication
(10) Pub. No.: US 2018/0147090 A1
(43) Pub. Date: May 31, 2018

(54) Total Eye Mask System

(71) Applicant: Linda Denison, Castle Hayne, NC (US)

(72) Inventor: Linda Denison, Castle Hayne, NC (US)

(21) Appl. No.: 15/362,526

(22) Filed: Nov. 28, 2016

(51) Int. Cl. A61F 9/04; A61F 11/12

(52) U.S. Cl. CPC A61F 9/04; A61F 11/12

(57) ABSTRACT

An apparatus comprising a sleep mask eye cover to reduce effects of ambient light for a sleeper; an elastic band to secure the apparatus to the sleeper during operation; a pair of earplug leashes comprising a first end and second end, wherein the pair of earplug leashes are fixedly attached via the first end to the elastic band at a first and second position adjacent to the sleeper's ears; and a pair of ear plugs removably coupled to the second end of the pair of earplug leashes, wherein the pair of ear plugs may be inserted into the sleeper's ears to reduce ambient noise.

### CROSS-REFERENCE TO RELATED APPLICATIONS

N/A

### BACKGROUND OF THE INVENTION

### Field of the Invention

The present invention relates generally to the field of non-medical sleep aids, and more specifically relates to a total eye mask systems.

### Background of the Invention

Many people have difficulty falling asleep and staying asleep. Light and noise are two of the main reasons people have challenges with sleeping. Ambient light can be blocked by use of room darkening curtains and the like. Use of a fan or a white noise generator can assist with blocking noise. However, sometimes the habits of a sleeping partner, such as watching TV, can't be mitigated by use of room darkening curtains or a white noise generator. A fan or white noise generator may interfere with the TV sound, making the partner turn up the volume of the TV to be heard over the noise of the fan or white noise generator, thus defeating the purpose of using the fan or white noise generator. To block the light of a television, a sleeper may place a pillow over their eyes, but the pillow may slip away from the eyes, or the pillow may cause the sleeper's head to get too hot. In some cases when ease of sleep is wanted, medications can be prescribed, but not everyone desires a medicated solution because of the potential for negative side effects. A suitable solution is desired.

Various attempts have been made to solve problems found in non-medical sleep aid art. Among these are found in: U.S. Patent and Publication Nos. 2014/0041091 to Sternlight; U.S. Pat. No. 5,940,886 to Smith; U.S. Pat. No. 2,537,768 to Laporte; and U.S. Pat. No. 5,058,606 to Malkoff. This prior art is representative of means to help sleepers fall asleep and stay asleep.

None of the above inventions and patents, taken either singly or in combination, is seen to describe the invention as claimed. Thus, a need exists for a reliable total eye mask system, and to avoid the above-mentioned problems.

### BRIEF SUMMARY OF THE INVENTION

In one embodiment of the present invention, an apparatus is provided comprising a sleep mask eye cover to reduce effects of ambient light for a sleeper; an elastic band to secure the apparatus to the sleeper during operation; a pair of earplug leashes comprising a first end and second end, wherein the pair of earplug leashes are fixedly attached via the first end to the elastic band at a first and second position adjacent to the sleeper's ears; and a pair of ear plugs removably coupled to the second end of the pair of earplug leashes, wherein the pair of ear plugs may be inserted into the sleeper's ears to reduce ambient noise.

In one embodiment, the sleep mask eye cover is constructed from a single piece of fabric material. In another embodiment, the elastic band comprises a plurality of pockets configured to accept the pair of earplugs for storage when not in use.

### BRIEF DESCRIPTION OF THE SEVERAL VIEWS OF THE DRAWINGS

It is to be understood that the drawings are to be used for the purposes of exemplary illustration only and not as a definition of the limits of the invention. Throughout the disclosure, the word “exemplary” is used exclusively to mean “serving as an example, instance, or illustration.” Any embodiment described as “exemplary” is not necessarily to be construed as preferred or advantageous over other embodiments. Referring to the drawings in which like reference character(s) present corresponding part(s) throughout:

FIG. 1A is an exemplary perspective front view of a total eye mask system according to an embodiment of the present invention.

FIGS. 1B-C are exemplary details views of an earplug component of a total eye mask system according to an embodiment of the present invention.

FIG. 1D is an exemplary perspective side view of a total eye mask system according to an embodiment of the present invention.

FIG. 1E is an exemplary perspective top view of a total eye mask system according to an embodiment of the present invention.

### DETAILED DESCRIPTION OF THE INVENTION

The detailed description set forth below in connection with the appended drawings is intended as a description of presently preferred embodiments of the invention and is not intended to represent the only forms in which the present invention may be constructed and or utilized.

The present invention advantageously fills the aforementioned deficiencies by non-medical sleep aids. The present invention is superior to other systems in that it effectively helps sleepers fall asleep and stay asleep.

A total eye mask system consisting of a combination sleep mask with attached ear plugs is disclosed herein. A sleep mask can be worn to block ambient light. The sleep mask is slipped over and worn around a person's head, as one piece, covering the eyes, ears, top portions of the nose, as well as portions of the sides and back of the head. Earplugs are removably inserted into a user's ear canals to reduce ambient noise from reaching the eardrums. The total eye mask system may be made of a sleep mask, an elastic band, a pair of earplugs, and a pair of earplug leashes. The pair of earplug leashes may be fixedly attached to the band of the eye mask above the ear area. When the mask is being used, the pair of earplugs may be easily accessed by reaching above the ear, grasping the earplug and inserting it in to the ear canal. When not in use, the pair of earplugs may be stored in a pocket on the elastic band. The pair of earplugs may be removably coupled to the pair of earplug leashes to allow replacement when needed.

Additional embodiments of the present invention may include use of plastic materials to make the earplugs. In addition, the eye mask may be made of materials to promote therapeutic use.

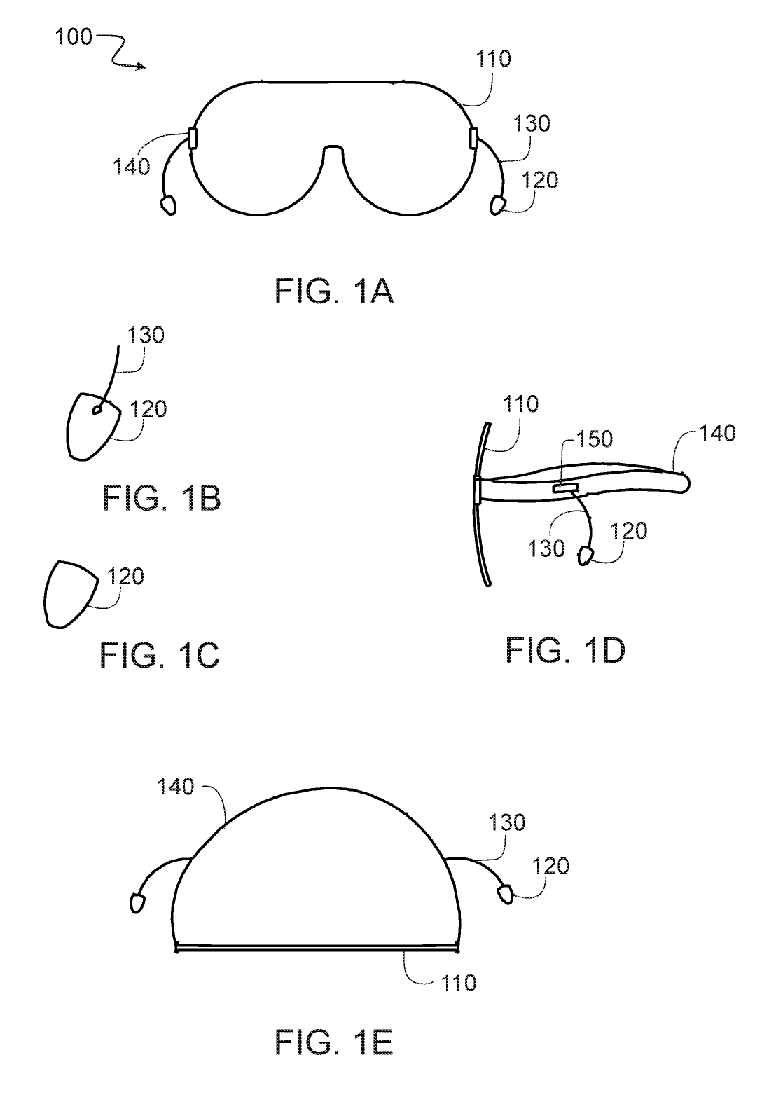

Referring now to FIGS. 1A-E, a total eye mask systems 100 is illustrated. The total eye mask system comprises a sleep mask eye cover 110, a pair of ear plugs 120, a pair of earplug leashes 130, and an elastic band 140. It is a particular advantage of the present invention to reduce the effects of ambient light and noise for a sleeper. The sleep mask may eye cover comprise fabric materials to promote ambient light blocking while providing comfort.

In one embodiment, the elastic band may comprise a garterized strap to be worn around the sleeper's head. In one embodiment, the elastic band may comprise a plurality of pockets 150. The plurality of pockets may be located proximate to the sleeper's ears during operation. The plurality of pockets may be configured to accept an earplug for storage, when not in use. The pair of earplug leashes may comprise a length suitable for retaining an earplug in a convenient position for the sleeper. In one embodiment, the pair of earplug leashes may further comprise a first-end and a second-end. The first-end of the earplug leash may comprise stitching to fixedly attach the earplug leash to the elastic band. The second-end of the earplug leash may comprise a removably coupleable earplug. This allows the earplugs to be replaced when needed.

It should be understood that the total eye mask system may be used in various conditions and locations. The total eye mask system may be used during daytime and nighttime. The total eye mask system may be used in a home environment, a hospital-type environment, and a transportation environment such as an airplane or a train. The exact specifications, materials used, and method of use of the total eye mask system may vary upon manufacturing.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

In addition, reference to “first,” “second,” “third,” and etc. members throughout the disclosure (and in particular, claims) are not used to show a serial or numerical limitation but instead are used to distinguish or identify the various members of the group.

What is claimed is:

1. An apparatus comprising:
a sleep mask eye cover to reduce effects of ambient light for a sleeper;
an elastic band to secure the apparatus to the sleeper during operation;
a pair of earplug leashes comprising a first end and second end, wherein the pair of earplug leashes are fixedly attached via the first end to the elastic band at a first and second position adjacent to the sleeper's ears; and
a pair of ear plugs removably coupled to the second end of the pair of earplug leashes, wherein the pair of ear plugs may be inserted into the sleeper's ears to reduce ambient noise.

2. The apparatus of claim 1, wherein the sleep mask eye cover is constructed from a single piece of fabric material.

3. The apparatus of claim 1, wherein the elastic band comprises a plurality of pockets configured to accept the pair of earplugs for storage when not in use.
